(19) United States
(12) Patent Application Publication
(10) Pub. No.: US 2014/0147519 A1
(43) Pub. Date: May 29, 2014

(54) Migraine Treatment

(71) Applicant: Larry A. MCPHAIL, Fowler, CA (US)

(72) Inventor: Larry A. MCPHAIL, Fowler, CA (US)

(21) Appl. No.: 14/038,332

(22) Filed: Sep. 26, 2013

Related U.S. Application Data
(60) Provisional application No. 61/729,865, filed on Nov. 26, 2012.
(60) Provisional application No. 61/804,845, filed on Mar. 25, 2013.

(51) Int. Cl. A61K 36/906; A61K 33/06; A61K 31/525; A61K 31/365; A61K 31/352; A61K 31/12; A61K 36/28; A61K 31/675

(52) U.S. Cl. CPC A61K 36/906; A61K 36/28; A61K 33/06; A61K 31/675; A61K 31/365; A61K 31/352; A61K 31/12; A61K 31/525
USPC 424/682

(57) ABSTRACT

A migraine treatment formulation is designed to be administered upon onset of migraine symptoms. The migraine treatment formulation is able to reduce the nausea and pain associated with a migraine headache through a pharmaceutical composition that carries a reduced chance of causing digestive discomfort. The treatment formulation accomplishes this through a combination of organic and inorganic agents, administered orally, that are optimized for absorption through the mucosal membrane and the lining of the stomach. The higher absorption rate of the active constituents quickly alleviates the various symptoms associated with a migraine headache while preventing unabsorbed constituents from causing digestive issues.

The current application claims a priority to U.S. Provisional Patent application Ser. No. 61/729,865 filed on Nov. 26, 2012 and U.S. Provisional Patent application Ser. No. 61/804,845 filed on Mar. 25, 2013.

### FIELD OF THE INVENTION

The present invention relates generally to a migraine treatment, more specifically to a fast acting orally administered mixture comprising a particular combination of organic extracts and chemical elements designed to reduce nausea symptoms, associated pain, and duration of migraine headaches.

### BACKGROUND OF THE INVENTION

Migraines are characterized as recurring severe headaches that are associated with autonomic symptoms. Migraine symptoms generally comprise symptoms that include but are not limited to nausea, vomiting, photophobia, phonophobia, and a severe pulsating headache on one or both sides of an individual's head. Typically migraine symptoms can last from 2 to 72 hours and are known to affect almost 30 million American. While the exact physiological interactions that lead to a migraine are not known, treatments have been developed that incorporate a plurality of organic compounds, inorganic compounds, and atomic elements in various therapeutic and pharmacological approaches that have been able to effectively prevent or treat migraine headaches.

One such therapeutic and pharmacological approach that has shown significant promise is the use of magnesium, specifically magnesium compounds, as an active ingredient to a plurality of orally administered migraine treatments. This approach has gained popularity due to several studies which have shown reduced magnesium levels in patients suffering from chronic migraine headaches. While magnesium compounds show promise current pharmacological approaches utilize magnesium compounds or magnesium salts that take a long time to be absorbed into a patient's blood stream. This slower absorption rate is due to the dissociation rate of the magnesium compounds and salts which are affected by several factors that include the acidity of the patient's stomach. Having a slower absorption rate is associated with several disadvantages. One disadvantage is that due to the slower absorption rate pharmacological approaches using magnesium compounds and salts are most effect as preventative treatments. Another disadvantage associated with the decreased absorption rate of magnesium compounds and salts is the digestive discomfort experienced by several patients who are unable to absorb a significant amount of the magnesium compound in through their stomach lining and mucosal membrane.

It is therefore the object of the present invention to provide a fast acting migraine treatment that is designed to be administered upon onset of migraine symptoms. The present invention is able to reduce the nausea and pain associated with a migraine headache through a pharmaceutical composition that carries a reduced chance of causing digestive discomfort. The present invention accomplishes this through a combination of organic and inorganic agents, administered orally, that are optimized for absorption through the mucosal membrane and the lining of the stomach. The higher absorption rate of the active constituents quickly alleviates the various symptoms associated with a migraine headache while preventing unabsorbed constituents from causing digestive issues.

### BRIEF DESCRIPTIONS OF THE DRAWINGS

FIG. 1 is an image displaying the molecular structure of parthenolide, an active constituent in the current embodiment of the present invention.

FIG. 2 is an image displaying the molecular structure of tanetin, an active constituent in the current embodiment of the present invention.

FIG. 3 is an image displaying the molecular structure of gingerol, an active constituent in the current embodiment of the present invention.

FIG. 4 is an image displaying the molecular structure of pyridoxal phosphate, an active constituent in the current embodiment of the present invention.

FIG. 5 is a flow chart diagram illustrating the method steps for administering the migraine treatment formulation as per the current embodiment of the present invention.

### DETAIL DESCRIPTIONS OF THE INVENTION

All illustrations of the drawings are for the purpose of describing selected versions of the present invention and are not intended to limit the scope of the present invention.

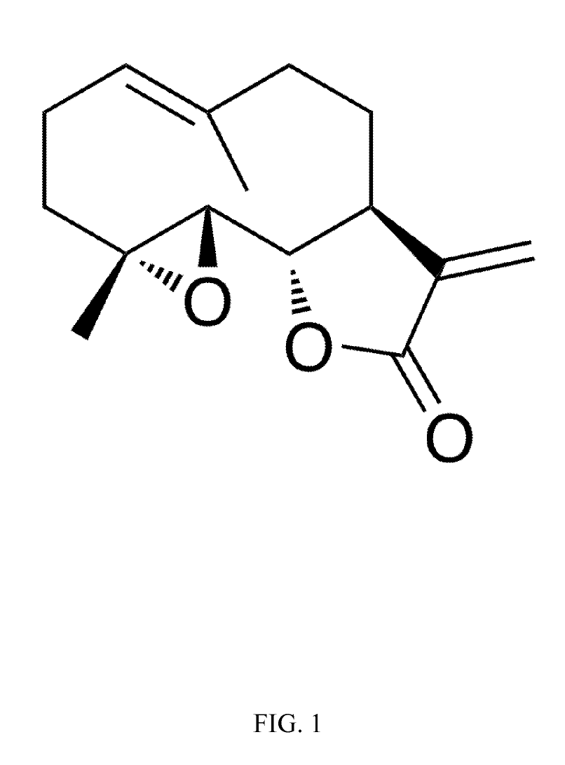

Referencing FIG. 1-4, a migraine treatment formulation is a fast acting orally administered composition that reduces the symptoms associated with a migraine within a few minutes of administration. Upon sensing the onset of a migraine, a user would ingest the migraine treatment formulation in order to reduce symptoms of nausea and pain associated with a migraine. The migraine treatment formulation additionally reduces the duration of a migraine. The migraine treatment formulation comprises feverfew extract, ginger extract, a soluble magnesium compound, and pyridoxal phosphate. The feverfew extract is derived from Tanecetum parthenium and contains organic compounds that function as anti-inflammatory and anti-analgesic agents. The ginger extract is derived from Zingiber officinale and contains organic compounds that functions as antiemetic agents. The soluble magnesium compound is provided as a source of magnesium that is absorbed through a user's stomach lining and doesn't cause gastrointestinal problems. Pyridoxal phosphate is the active form of vitamin B₆ that functions as a coenzyme in crucial metabolic and catabolic reactions associated with migraine symptoms. It should be noted that the migraine treatment formulation is provided as an aqueous solution that contains the feverfew extract, the ginger extract, the soluble magnesium compound, and the pyridoxal phosphate in specific quantities.

TABLE 1

| | Quantity Range Present | Physiological |
|---|---|---|
| Component | within Formulation | Effect |
| Feverfew | 0.5 grams to | Anti-inflammatory |
| Extract | 1.5 grams | |
| Ginger | 0.5 grams to | Antiemetic |
| Extract | 1.5 grams | |
| Soluble Magnesium | 0.1 grams to | Magnesium Supplement |
| Compound | 10.0 grams | |
| Pyridoxal Phosphate | 0.1 grams to | Vitamin B₆ Supplement |
| | 10.0 grams | |

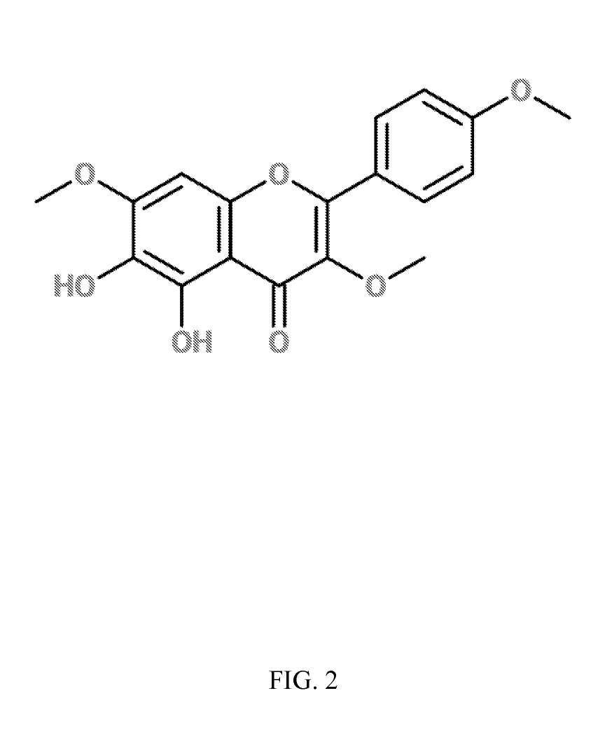

Referencing FIG. 1 and FIG. 2, the feverfew extract is provided as a pain relieving agent. The feverfew extract utilized in the migraine treatment formulation is derived using existing separation techniques used on T. parthenium. The feverfew extract is generated through an extraction process that utilizes the flowers and fruit bodies of T. parthenium. The feverfew extract is found ranging between 0.5 grams to 1.5 grams of the migraine treatment formulation. In current embodiment of the present invention, a dosage of the migraine treatment formulation contains approximately 1.0 grams of the feverfew extract. The feverfew extract contains parthenolide and tanetin. Parthenolide and tanetin are the active constituents of the feverfew extract. Through the various interactions, the active constituents of the feverfew extract provide the feverfew extract as a pain relieving agent.

TABLE 2
Feverfew Extract

| Components | Quantity Range in Extract | Actual Quantity |
|---|---|---|
| Parthenolide | 0.1% to 3.0% wt | 0.4% wt |
| Tanetin | 0.1% to 10.0% wt | 6.0% wt |

Referencing FIG. 1, Parthenolide is a sesquiterpene lactone of the germacranolide class of organic molecules that occur naturally in T. parthenium. Parthenolide function primarily as an anti-inflammatory and an anti-hyperalergesic. Parthenolide is found ranging between 0.1% and 3.0% wt of the feverfew extract. In the preferred embodiment of the present invention, the amount of parthenolide is 0.4 wt % of the feverfew extract.

TABLE 3
Parthenolide

| | |
|---|---|
| CAS NO. | 20554-84-1 |
| IUPAC | (1aR,7aS,10aS,10bS)-1a,5-dimethyl-8- |
| | methylene-2,3,6,7,7a,8,10a,10b- |
| | octahydrooxireno[9,10]cyclodeca[1,2-b]furan- |
| | 9(1aH)-one |
| Molar Mass | 248.32 g/mol |
| Physiological Effects | Anti-Inflammatory |
| | Anti-hyperalergesic |

Referencing FIG. 2, Tanetin is a lipophilic flavonol class of organic molecules that occur naturally in the T. parthenium. Tanetin functions primarily as an anti-inflammatory. Tanetin is found ranging between 0.1% and 10.0% wt of the feverfew extract. In the preferred embodiment of the present invention, tanetin is 6.0% wt of the feverfew extract.

TABLE 4
Tanetin

| | |
|---|---|
| PubChem Substance ID | 85296543 |
| IUPAC | 6-hydroxykaempferol 3,7,4′-trimethyl ether |
| Molar Mass | 344.08 g/mol |
| Physiological Effects | Anti-Inflammatory |

The ginger extract is provided as an antiemetic agent. Antiemetic agents are effective at reducing feeling of nausea and vomiting. The ginger extract utilized in the migraine treatment formulation is derived using existing separation techniques currently used on Z. officinale. In the current embodiment of the present invention the ginger extract is found ranging between 0.5 grams to 1.5 grams of the migraine treatment formulation. The ginger extract contains gingerol. Gingerol functions as the active constituent of the ginger extract.

TABLE 5
Ginger Extract

| Components | Quantity Range in Extract | Actual Quantity |
|---|---|---|
| Gingerol | 0.1% to 20.0% wt | 5.0% wt |

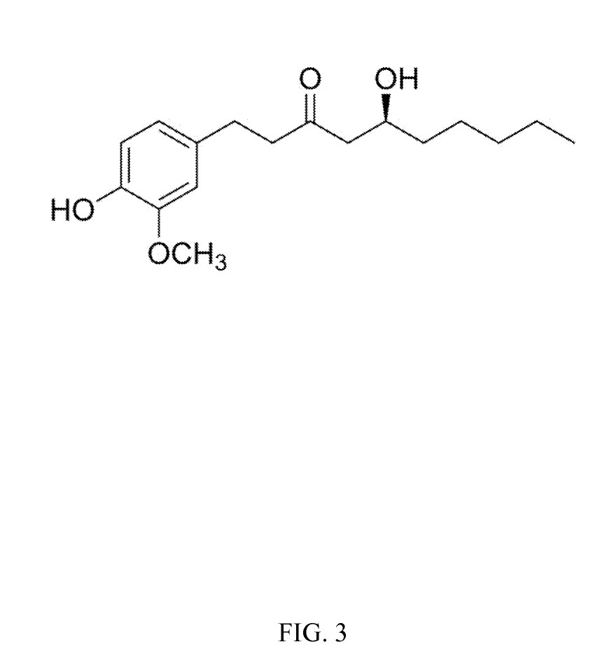

Referencing FIG. 3, Gingerol is also known as [6]-gingerol is an organic molecule, similar to capsaicin, which occurs naturally in the Z. officinale. Gingerol function primarily as an antiemetic in the migraine treatment formulation. The amount of gingerol found in the ginger extract can vary dependent on the plant and the extraction method. In the current embodiment of the present invention, gingerol is found in ranging between 0.1% and 20.0% wt of the ginger extract. In the preferred embodiment of the present invention, gingerol is 5.0% wt of the ginger extract.

TABLE 6
Gingerol
[6]-gingerol

| | |
|---|---|
| CAS NO. | 1391-73-7; 23513-14-6 |
| IUPAC | (S)-5-hydroxy-1-(4-hydroxy-3- |
| | methoxyphenyl)-3-decanone |
| Molar Mass | 294.38 g/mol |
| Physiological Effects | Antiemetic |

The soluble magnesium compound is provided as a source of magnesium, Mg²⁺ that is rapidly absorb through the lining of the user's stomach. Migraine sufferers have been linked with magnesium deficiencies that affect the onset and duration of migraine symptoms. By providing a supplemental source of magnesium in a fast acting orally administered formulation, the present invention is able to reduce or stop the onset of migraine symptoms within minutes of administering a dosage. The soluble magnesium compound is provided in a manner that reduces gastrointestinal distress commonly associated with the consumption of the magnesium. The soluble magnesium compound accomplishes through a composition that facilitates the absorption rate of magnesium across the stomach lining preventing large amount of magnesium from entering into the small intestine. In the current embodiment of the present invention, the soluble magnesium compound is found ranging between 0.1 grams to 10.0 grams of the formulation. In the preferred embodiment of the present invention, a dosage of the migraine treatment formulation contains 130 mg to 260 mg of dissolved magnesium. It should be noted that the soluble magnesium compound may comprise magnesium containing composition that include but are not limited to, magnesium oxide, magnesium chloride, magnesium citrate, magnesium gluconate, magnesium glycinate, magnesium threonate, and magnesium stearate as well as any magnesium salt derived utilizing an amino acid constituent.

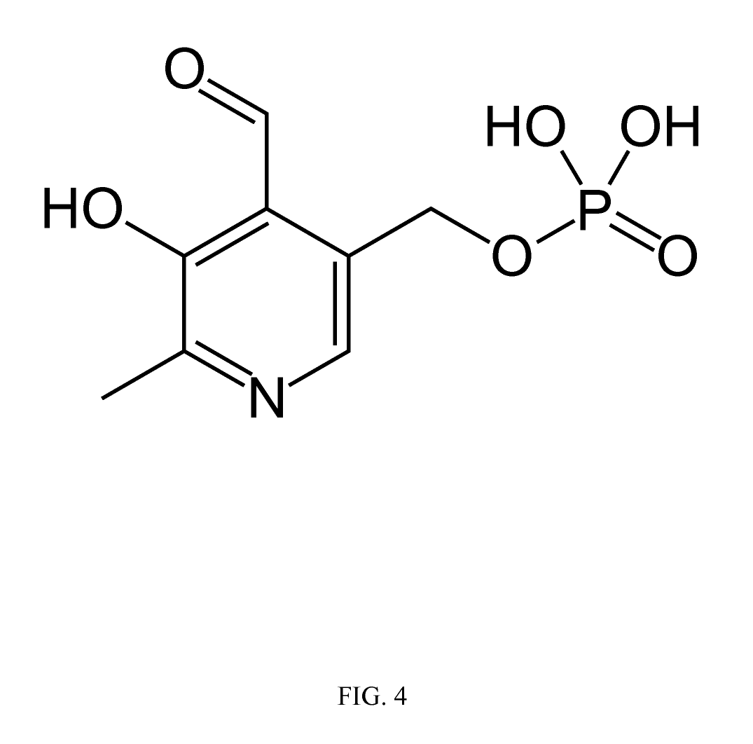

Referencing FIG. 4, Pyridoxal phosphate is provided as a supplement that that assists in crucial metabolic and catabolic reactions associated with migraine symptoms. Pyridoxal phosphate is the active form of Vitamin B₆. Pyridoxal phosphate is derived from pyridoxine and is involved in the catalysis of glycogen and glucose. Pyridoxal phosphate has been determined to treat anemia caused by a pyridoxine deficiency which has been linked with migraines in some users. In the current embodiment of the present invention, pyridoxal phosphate is found ranging between 0.1 grams to 10.0 grams of the formulation.

TABLE 7
Pyridoxal Phosphate

| | |
|---|---|
| CAS NO. | 54-47-7 |
| IUPAC | Pyridoxal 5-phosphate, PAL-P, PLP, |
| | Vitamin B6 phosphate |
| Molar Mass | 247.142 g/mol |

In an additional embodiment of the present invention, the migraine treatment formulation additionally comprises riboflavin. Riboflavin is commonly known as vitamin B2 and has been found to improve magnesium transport across the mitochondrial membrane. In the additional embodiment of the present invention, riboflavin is found ranging between 1.0 grams to 10.0 grams of the formulation.

In an additional embodiment of the present invention the current mixture can include a buffering agent. The buffering agent would function as a compound or substance that minimizes changes in pH while limiting the effects of dissociation rates of the active constituents. Buffering agents that could potentially be utilized by the present invention can include but are not limited to organic salts, ionic compounds, and chemical or organic mixtures as well as any combination thereof.

In an additional embodiment of the present invention, additives can be included to improve the flavor of the mixture. While the current embodiment of the present invention does not contain flavor agents, additional embodiments can include flavor enhancers that would improve the palatability of the current mixture. Flavor agents that could be used with the current mixture include but are not limited to, sweeteners, artificial fruit flavors, natural fruit flavors, as well as any compound or substance that alters the palatability of the current mixture while having minimal influence on the active ingredients.

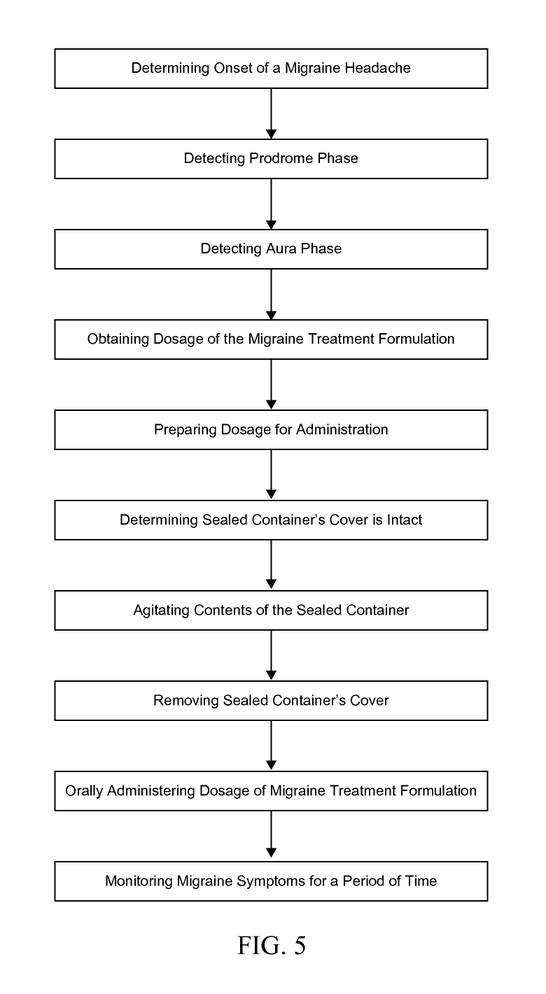

Referencing FIG. 5, the method of administering the migraine treatment formulation comprises the steps of determining the onset of a migraine headache; obtaining a dosage of the migraine treatment formulation; preparing the dosage of the migraine treatment formulation for administration; administering the prepared dosage of the migraine treatment formulation; and monitoring migraine symptoms for a period of time. The determination of the onset of a migraine is provided as an initiating step. Following the determination, the user would proceed to obtain a dosage of the migraine treatment formulation. The user would then prepare the migraine treatment formulation to ensure its effectiveness at treating the migraine symptoms. With the migraine treatment formulation prepared for administration, the user would ingest the migraine treatment formulation. User would monitor their migraine symptoms to in order to determine the effectiveness of the dosage. The migraine treatment formulation is designed to be fast acting treatment that reduces the symptoms associated with a migraine within a period of time ranging between 10 to 50 minutes.

TABLE 8

| | Quantity per | Active | Conc. | Physiological |
|---|---|---|---|---|
| Component | Dosage | Constituents | (wt %) | Effect |
| Feverfew | 1.0 g | Parthenolide | 0.4% | Anti- |
| | | | | inflammatory |
| Extract | | Tanetin | 6.0% | Anti- |
| | | | | inflammatory |
| Ginger | 1.0 g | Gingerol | 5.0% | Antiemetic |
| Extract | | | | |
| Soluble | 1.0 g to 10.0 g | Magnesium | 100% | Magnesium |
| Magnesium | | Mg²⁺ | | Supplement |
| Compound | | | | |
| Pyridoxal | 1.0 g to 10.0 g | Pyridoxal | 100% | Vitamin B₆ |
| Phosphate | | Phosphate | | Supplement |

The step of determining the onset of a migraine headache is provided as the determinant step that initiates the treatment method. The migraine treatment formulation is most effective at reducing and stopping symptoms associated with a migraine during the initial migraine phases. Migraines occur in four phases the prodrome phase, the aura phase, the pain/headache phase, and the postdrome phase. the prodrome phases occurs hours or days before pain/headache phase and is characterized by a wide variety of phenomena that include but are not limited to, altered mood, irritability, depression or euphoria, fatigue, craving certain foods, stiff muscles, constipation or diarrhea, and sensitivity to smell or noises. The aura phase immediately precedes the pain/headache phase and is characterized by the visual distortion of sensitivity to light. The pain/headache phase is classic unilateral headache that characterizes a migraine. The postpodrome phase can last for several days following a migraine and is characterized by migraine symptoms that persist following the main migraine headache. By detecting the prodrome phase and/or the aura phase, the user can take the necessary steps to administer the migraine treatment formulation.

The step of obtaining a dosage of the migraine treatment formulation is provided as the step proceeds the early determination of a migraine headache. The migraine treatment formulation is provided as a dosage contained within a sealed container. The dosage is in solution form and requires a container to allow for facilitated transport and storage. The sealed container retains the solution within a cavity which can be accessed by removing a cover. A dosage of the migraine treatment formulation comprises 1.0 grams of the feverfew extract, 1.0 grams of ginger extract, an amount ranging between 130 mg to 260 mg of magnesium Mg²⁺, and pyridoxal phosphate ranging between 1.0 g to 10.0 g of the solution.

The step of preparing the dosage of the migraine treatment formulation is provided in order to ensure the effectiveness of the migraine treatment formulation. After obtaining the sealed container, the user would inspect the cover of the sealed container to ensure that it is intact. If the cover is intact than the user is assured that the contents are in the sealed container and have not been tampered with. The user would then agitate the sealed container to ensure the proper dissolution of the dosage. The user would then remove the sealed container's cover allowing access to the solution. The dosage would then be orally ingested by the user. The step of monitoring the migraine symptoms for a period of time occurs following the ingestion of the migraine treatment formulation. The user would monitor their symptoms for a period of time that ranges within 10 to 50 minutes. During this period of time the user's migraine symptoms would subside.

Although the invention has been explained in relation to its preferred embodiment, it is to be understood that many other possible modifications and variations can be made without departing from the spirit and scope of the invention as hereinafter claimed.

What is claimed is:

1. A migraine treatment formulation comprises:
feverfew extract;
ginger extract;
a soluble magnesium compound, wherein the soluble magnesium dissolves forming Mg²⁺;
pyridoxal phosphate;
the feverfew extract comprises parthenolide and tanetin;
the ginger extract comprises gingerol;
the feverfew extract is found ranging between 0.5 g to 1.5 g of the formulation;
the ginger extract is found ranging between 0.5 g to 1.5 g of the formulation;
the soluble magnesium compound is found ranging between 0.1 g to 10.0 g of the formulation;
pyridoxal phosphate is found ranging between 1.0 g to 10.0 g of the formulation;
parthenolide is found ranging between 0.1% to 3.0% wt of the of the feverfew extract;
tanetin is found ranging between 0.1% to 10.0% wt of the feverfew extract; and
gingerol is found ranging between 0.1% to 20.0% wt of the ginger extract.

2. The migraine treatment formulation as claimed in claim 1 additionally comprises riboflavin.

3. The migraine treatment formulation in claim 2 wherein riboflavin is found ranging between 1.0 g to 10.0 g of the formulation.

4. The migraine treatment formulation in claim 1 wherein parthenolide is found at a concentration of 0.4% wt of the feverfew extract.

5. The migraine treatment formulation in claim 1 wherein tanetin is found at a concentration of 6.0% wt of the feverfew extract.

6. The migraine treatment formulation in claim 1 wherein gingerol is found at a concentration of 5.0% wt of the ginger extract.

7. The migraine treatment formulation in claim 1 wherein magnesium is found ranging between 130 mg and 260 mg of the formulation.

8. The method of administering a migraine treatment formulation comprises the steps of:
providing a dosage of the migraine treatment formulation within a sealed container;
determining the onset of a migraine headache;
obtaining the dosage of the migraine treatment formulation;
preparing the dosage of the migraine treatment formulation for administration;
administering the dosage of the migraine treatment formulation, wherein the dosage is orally ingested; and
monitoring migraine symptoms for a period of time until is the determined that the migraine symptoms have subsided.

9. The step of determining the onset of the migraine headache in claim 8 comprises the steps of:
providing a migraine headache comprising the a prodrome phase and an aura phase;
detecting prodrome phase associated with the onset of the migraine headache; and
detecting aura phase associated with the onset of the migraine headache.

10. The step of obtaining the dosage of the migraine treatment formulation in claim 8 additionally comprises the steps of:
obtaining the sealed container holding containing a solution comprising 1.0 grams of feverfew extract, 1.0 grams of ginger extract, an amount of a soluble sodium compound, and an amount of pyridoxal phosphate;
wherein the amount of the soluble sodium compound comprises magnesium Mg²⁺ ranging between 130 mg to 260 mg in the solution; and
wherein the amount of the pyridoxal phosphate is found ranging between 1.0 g to 10.0 g of the solution.

11. The step of preparing the dosage of the migraine treatment formulation in claim 8 additionally comprises the steps of:
determining the sealed container's cover is intact;
agitating the sealed container in order to ensure dissolution of the dosage; and
removing the sealed container's cover.

12. The step of monitoring the migraine symptoms in claim 8 wherein the period of time for the migraine symptoms to subside ranges between 10 to 50 minutes.
